(19) United States
(12) Patent Application Publication
(10) Pub. No.: US 2015/0011840 A1
(43) Pub. Date: Jan. 8, 2015

(54) EVENT SEQUENCING USING ACOUSTIC RESPIRATORY MARKERS AND METHODS

(71) Applicant: ISONEA (ISRAEL) LIMITED, Haifa (IL)

(72) Inventor: Noam Gavriely, Haifa (IL)

(21) Appl. No.: 14/236,644

(22) PCT Filed: Aug. 7, 2012
(86) PCT No.: PCT/IL2012/050294

§ 371 Date: Sep. 24, 2014

(30) Foreign Application Priority Data
Aug. 8, 2011 (AU) 2011903164

(51) Int. Cl. A61B 7/00; A61B 5/11; A61B 5/00; A61B 5/08

(52) U.S. Cl. CPC A61B 7/003; A61B 5/0826; A61B 5/0823; A61B 5/1116; A61B 5/7282; A61B 5/742; A61B 5/7264
USPC 600/301

(57) ABSTRACT

Disclosed are systems and methods for detecting e.g. physiological, pathophysiological and therapeutic events, and characterizing the temporal relationship between these events and acoustic respiratory markers.

### FIELD OF THE INVENTION

The present disclosure relates to the field of respiratory acoustics. It relates particularly but not exclusively to methods and systems for monitoring physiological and/or pathophysiological and/or therapeutic parameters together with acoustic respiratory markers from a subject and characterizing a relationship, such as a temporal relationship, between one or more acoustic respiratory markers and e.g. a physiological event occurring in the subject.

### BACKGROUND TO THE INVENTION

The body comprises a complex interaction of physiological systems. Events such as physiological, pathophysiological, psychological and physical events can be explained in the context of the “system” involved. For example, a reflux event in a subject can be explained by reference to the digestive system. However, this event may influence or be influenced by other systems in the body such as, for example, the respiratory system.

Some such interactions are known to occur and can be explained by proven and well understood medical science. Other interactions are unproven but are accepted or at least believed to exist based on scientific theories or studies which are not yet fully understood. Quantitative analysis of the interaction has hitherto not been feasible and the interactions evaluated only in an empirical sense.

Methods exist for accurate detection of acoustic markers, such as those disclosed in U.S. Pat. No. 6,168,568 issued Jan. 2, 2001, to Gavriely entitled “Phonopneumograph System”, U.S. Pat. No. 6,261,238 issued Jul. 17, 2001, to Gavriely entitled “Phonopneumograph System”, and Breath Sounds Methodology (N. Gavriely, Florida: CRC Press, Inc., 1995), which allows quantitative assessment of breath sounds and timing. Other methods such as those disclosed in U.S. Pat. No. 7,347,824 issued Mar. 25, 2008, to Wilkinson et al. for “Method and Apparatus for Determining Conditions of Biological Tissues,” involve use of an introduced signal.

Methods also exist for detection of physiological and pathophysiological events. Examples include the onset and end of apnea in a patient suffering from e.g. sleep apnea syndrome, detection of a breath or forced exhalation, determination of a change of settings of the ventilator in a patient being artificially ventilated, or the occurrence of reflux event to name a few. Additionally, means exist to detect a posture change (e.g. movement from upright to supine or left-to-right lateral decubitus) and the onset of physical activity. In addition, the timing of administration of a medication and the time of onset and dynamics of its effect can be ascertained using known methods.

This disclosure uses methods for detecting e.g. physiological, pathophysiological and therapeutic events, and characterizing the temporal relationship between these events and acoustic respiratory markers.

This background discussion, including reference to documents, acts, materials, devices, articles and the like is intended to explain the context of the present disclosure. This discussion is not to be taken as an admission or a suggestion that any of the material referred to was published, known or part of the common general knowledge as of the priority date of any of the claims.

### SUMMARY OF THE INVENTION

Acoustic respiratory markers (ARMs) often coincide with other physiological, pathophysiological and therapeutic events such as upper-airway closure (sleep-apnea), respiratory maneuvers, respiration, step-change in airway pressure (artificial ventilation and continuous positive airway pressure (CPAP)) and reflux of gastric content into the esophagus (gastro-esophageal reflux disease). Such acoustic markers may include normal breath sound amplitudes, wheezes, other Continuous Adventitious Breath Sounds (CABS), coughs, snores and crackles to name a few. Even the most basic events such as eating and talking can trigger respiratory events.

The present disclosure provides a method for characterizing a temporal relationship between an acoustic respiratory marker and an event in a mammalian subject or patient. The method includes (a) simultaneously monitoring an acoustic signal from the respiratory system of the subject and at least one parameter selected from a group including a physiological parameter, a pathophysiological parameter, a patient-reported symptom and a therapeutic parameter associated with the subject; (c) identifying the event in the monitored parameter(s); and (d) identifying, in the monitored acoustic signal, the presence of one or more respiratory markers coinciding with and/or preceding and/or ensuing the event. The relationship is characterized by determining a temporal correlation between the event and the one or more acoustic respiratory markers.

In an aspect of the disclosure, the acoustic respiratory marker is selected from the group including but not limited to a signal indicative of a wheeze, cough, snore, crackle and a breath sound amplitude. The event is selected from a group including but not limited to: a reflux event, the onset or end of apnea, change of settings on a ventilator, a postural change, an indication of a symptom of a patient and administration of therapeutic agent or treatment.

Methods are configurable to include the step of introducing a sound signal having known acoustic characteristics into the respiratory system of the subject, and wherein the monitored acoustic signal includes the introduced sound signal after it has travelled through at least part of the respiratory system of the subject.

In another aspect of the disclosure, the method includes the step of representing the monitored acoustic signal for a time period using a mathematical model and evaluating one or more parameters of the model. The one or more evaluated parameters are quantitative indicators of the relationship between the event and the one or more acoustic markers. The time period commences during or after the event although it may also include a time period before the event in some embodiments.

One or more of the following may be used to characterize the relationship or the coefficients of a polynomial equation employed in a mathematical model or other representation of the monitored acoustic signal:

(a) time constant, τ determined using the relationship

$$y = A {e}^{- \frac{t}{τ}} ;$$

(b) variance, σ² as determined using the relationship

$${σ}^{2} = \frac{1}{N - 1} {\sum }_{i = 1}^{N} {( {x}_{i} - {x}_{0} )}^{2} ;$$

(c) skewness, γ₁ as determined using the relationship

$${γ}_{1} = \frac{1}{N} {\sum }_{i = 1}^{N} {( \frac{{x}_{i} - {x}_{0}}{σ} )}^{3} ;$$

(d) kurtosis, γ₂ as determined using the relationship

$${γ}_{2} = \frac{1}{N} {\sum }_{i = 1}^{N} {( \frac{{x}_{i} - {x}_{0}}{σ} )}^{4} ;$$

and
(e) Hill equation exponent, n as determined using the relationship

$$Θ = m \frac{{( t )}^{n}}{{( t )}^{n} + {( {T}_{50} )}^{n}} ;$$

where:
   y is the respiratory marker value;
   A and τ are constants;
   N is the number of acoustic signal data points;
   σ is the standard deviation;
   xᵢ is the value of data point i
   x₀ is the value of the data point corresponding to the event;
   θ is the fraction of the maximum data;
   T₅₀ is the time where the data is 50% of the maximum value;
   t is the time;
   n is an exponent representing acuteness of the change in respiratory marker; and
   m is a proportion coefficient.

In yet another aspect of the disclosure, the method includes the step of averaging data representing the acoustic signal for a plurality of events identified in the monitored parameter to characterize the relationship. An embodiment may also/alternatively include the step of determining the extent of the respiratory marker.

The method of characterization referred to above may be employed in a method for diagnosing a condition in the subject.

Viewed from another aspect, the present disclosure provides a system for characterizing a relationship between an acoustic respiratory marker from a subject and an event in a subject, the system including: (a) an acoustic monitor capable of monitoring an acoustic signal from the respiratory system of the subject; (b) a parameter monitor capable of monitoring one or more parameters selected from the group including a physiological parameter, a pathophysiological parameter, a therapeutic parameter associated with the subject and a patient-reported symptom; and (c) a processor incorporating: (i) an identifier module identifying the occurrence of the event in the monitored parameter(s); (ii) a marker module locating one or more acoustic markers in the acoustic respiratory signal coinciding with and/or preceding and/or ensuing the event; and (iii) a characterization module configured to characterize the relationship by determining a temporal correlation between the event and the one or more acoustic respiratory markers.

Viewed from still another aspect, the present disclosure provides a system for characterizing a relationship between an acoustic respiratory marker from a subject and an event in a subject, the system including: (a) an acoustic monitoring means for monitoring an acoustic signal from the respiratory system of the subject; (b) a parameter monitoring means for monitoring a parameter selected from the group including a physiological parameter, a pathophysiological parameter, a therapeutic parameter associated with the subject and a patient-reported symptom; and (c) a processing means incorporating: (i) an identifier module identifying the occurrence of the event in the monitored parameter(s); (ii) a marker module locating one or more acoustic markers in the acoustic respiratory signal coinciding with and/or preceding and/or ensuing the event; and (iii) a characterization module configured to characterize the relationship by determining a temporal correlation between the event and the one or more acoustic respiratory markers.

In still another aspect of the disclosure, the system includes a sound source for generating a sound signal having known characteristics and an introducer or means for introducing capable of introducing the sound signal to the respiratory system of the subject, wherein the acoustic monitor or acoustic monitoring means is capable of monitoring the introduced sound signal after the sound has travelled though at least part of the respiratory system of the subject. The system may also include a user interface presenting a graphical display of the monitored signals and receiving a user selection of a data window for further characterization.

In an embodiment, the characterizing module further characterizes the relationship between an acoustic respiratory marker from a subject and an event in a subject by calculating a mathematical model approximating at least a portion of the monitored acoustic signal. The characterizing module may also evaluate parameters of the mathematical model to quantify the characterization.

The acoustic respiratory marker may be selected from a group including but not limited to a signal indicative of a wheeze, cough, snore, crackle and a breath sound amplitude. The event may be selected from a group including but not limited to: a reflux event, the onset or end of apnea, change of settings on a ventilator, a postural change and administration of a therapeutic agent or treatment.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a block diagram illustrating steps in a method of characterizing a relationship between an event and an Acoustic respiratory markers (ARM), according to an embodiment of the disclosure;

FIG. 2 is a schematic illustration of aspects of a system for characterizing a relationship between an event and an ARM, according to an aspect of the disclosure;

FIG. 3 shows an example of a temporal correlation between a therapeutic event (administration of a bronchodilator) and the onset of a change in respiratory marker, being wheeze activity;

FIG. 4 shows an example of a temporal correlation between a pathophysiological event (e.g. a reflux event) and the onset of a change in respiratory marker, being cough activity. In this example, a cough-induced reflux event is shown;

FIG. 5 shows an example of a temporal correlation between a pathophysiological event (e.g. a reflux event) and the onset of a change in respiratory marker, being cough activity. In this example a reflux-induced cough is shown;

FIG. 6 shows an example of a temporal correlation between a pathophysiological event (e.g. a reflux event) and the onset of a change in respiratory marker, being wheeze activity. In this example, reflux-induced wheezes are shown;

FIG. 7 shows an example of a temporal correlation between a physiological event (e.g. change in posture) and the onset of a change in respiratory marker, being crackle activity. In this example, posture-induced crackles are shown;

FIG. 8 shows an example of a temporal correlation between a physiological event (e.g. administration of a diuretic medication) and the onset of a change in respiratory marker, being crackle activity; and

FIG. 9 shows a general example of temporal mapping of acoustic markers to a number of reflux events, the graph represents an average of multiple events with standard deviations shown above and below the average line.

### DETAILED DESCRIPTION

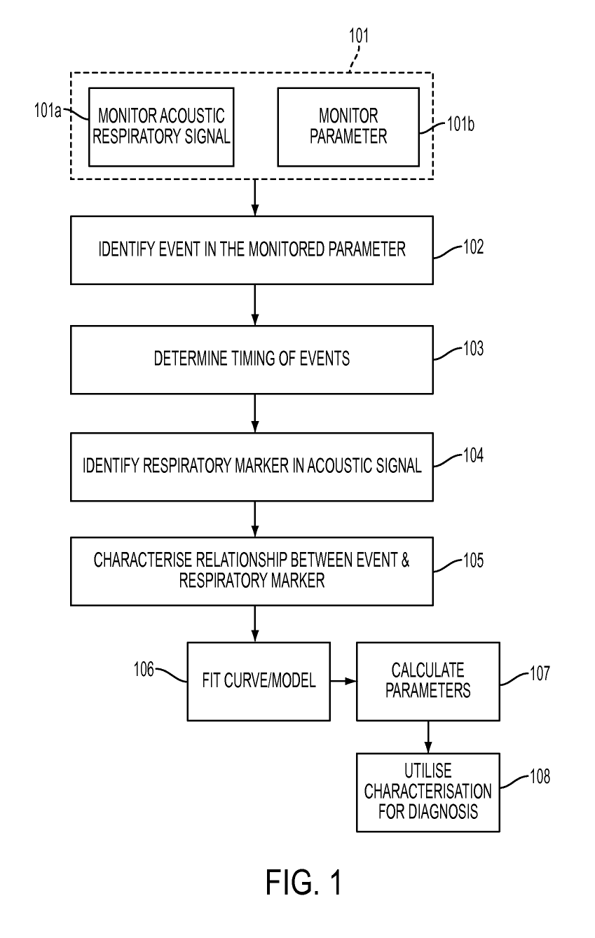

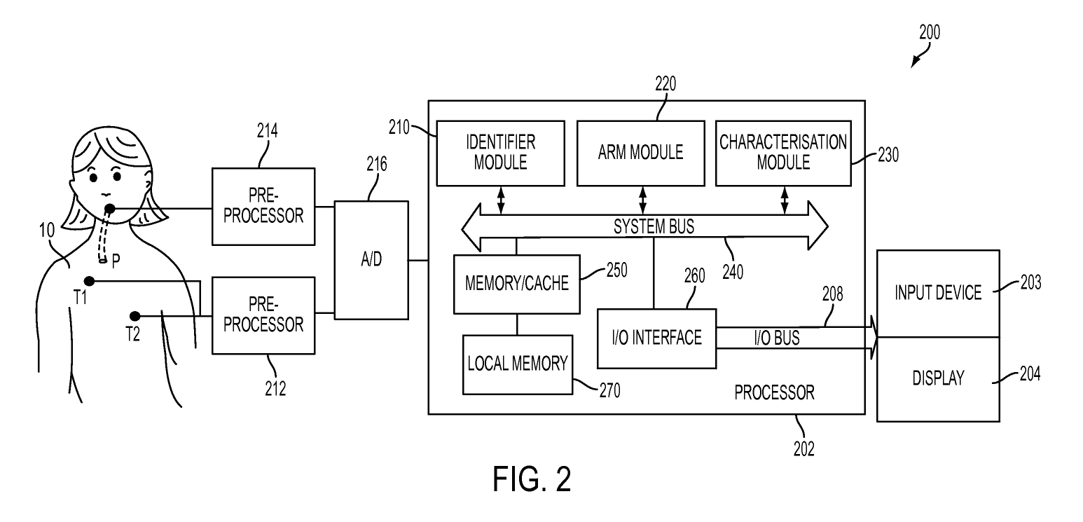

Referring firstly to FIG. 1, a flow diagram illustrates steps in a method for characterizing a relationship between e.g. a physiological event and one or more Acoustic respiratory markers (ARMs). In a step 101 an acoustic signal emanating from the respiratory system of the subject is monitored (101a) simultaneously with at least one physiological parameter from the subject or patient (101b). The one or more monitored parameter may be any physiological, pathophysiological, therapeutic, psychological or other parameter in which an event of interest can be identified during the monitored period. For example, the physiological parameter of esophageal pH may be monitored (see for example FIG. 2).

In a step 102 an event is identified in the monitored parameter. The event may be e.g. a physiological event, a pathophysiological event or a therapeutic event. The event may be identified manually, e.g. by a medical practitioner having regard to the monitored parameter over the monitored period. The event may also be monitored by the patient, or another healthcare provider. Alternatively, the event may be identified automatically, e.g. by a computer processor programmed (in hardware or software) to receive a signal representing the monitored parameter and identify events in that signal. Automated event identification may involve identification of parameter values exceeding (or alternatively falling below) a pre-set threshold. Alternatively, detection of a pattern in the monitored parameter which is indicative of an event (e.g. change of posture from sitting to lying supine as detected by pressure sensors in the subject's chair or bed) may be used. Other approaches to automatic identification of the event include indication of symptoms by the patient (e.g. onset or relief of pain, onset of dyspnea) or indication by a health care professional of the onset of a perturbation to the patient (e.g. onset of chest physical therapy, beginning of IV medication or Tracheal extubation/intubation).

In a step 103 the timing of the identified event within the monitored period is determined and in a step 104 one or more ARMs are identified in the acoustic respiratory signal monitored at 101a. ARMs may be detected in a period of time preceding the event, and in a period of time following the event and may be compiled. For example, Acoustic Markers may be compiled from 10 minutes prior to the event, to 10 minutes after the event although time periods as short as a few seconds or as long as a few hours before and after the event are also contemplated. If there are multiple occurrences of the event, the ARM value (or an allocated score) may be averaged according to the relative time of each marker in relation to the time of the event.

Identification of ARMs can be performed automatically or semi-automatically by a computer processor programmed in hardware or software to detect markers. Methods for detection of adventitious respiratory sounds (providing useful markers) such as wheezes, coughs, crackles, rhonchi and snores are disclosed in U.S. Pat. No. 6,261,238 issued Jul. 17, 2001, to Gavriely for “Phonopneumograph System,” and U.S. Pat. No. 6,168,568 issued Jan. 2, 2001, to Gavriely for “Phonopneumograph System.” Other methods are contemplated including, but not by any means limited to, methods disclosed in U.S. Pat. No. 7,347,824 issued Mar. 25, 2008, to Wilkinson et al. for “Method and Apparatus for Determining Conditions of Biological Tissues,” which involves introduction of a sound signal having known characteristics into the subject's airway and detection of the signal after it has passed through at least part of the respiratory system of the subject and calculating the transfer characteristics of the transmitted sound with respect to the timing of the event.

The one or more ARMs may be identified by analysis of the entire dataset obtained during the monitored period, or only a subset of data corresponding to a period preceding and/or ensuing the identified event. ARMs may be given a score based on the extent of the marker, or may have an inherent value (e.g. where the ARM is a breath sound amplitude). Analysis of the acoustic respiratory signal to identify one or more ARMs may be performed before the timing of events is determined, or after. When performed after, efficiencies may be obtained by only analyzing data windows in which an event has occurred. Thus, windows of acoustic respiratory data corresponding to a time period in which there are no events are not analyzed for detection of ARMs. In a step 105 the temporal relationship between the event and the one or more ARMs is characterized. This may involve comparing a pre-event extent of ARMs with the extent of ARMs in time periods following an event.

The ARMs may be characterized by curve fitting or mathematical modeling of the markers e.g. using a distribution function (step 106). The distribution function may represent the distribution around a single event or a mean of distributions around multiple events. The distribution function may be visually displayed in the form of a histogram plot, where acoustic markers are distributed according to the relative time of their occurrence. From this characterization, quantitative parameters may be calculated in a step 107 and used for diagnosis (at 108) and/or further analysis, clinical decision or the like. These parameters may be derived from the data using a mathematical function representing the distribution function, the average score of the acoustic markers before and/or after the event, and other characteristics of the distribution function such as the variance, skewness and kurtosis of the distribution curve.

Referring now to FIG. 2 there is shown a schematic illustration of components of a system for characterizing a relationship between an event (as described above) and an ARM. Acoustic monitors in the form of transducers T1 and T2 are capable of monitoring an acoustic signal from the respiratory system of the subject 10. The acoustic signal may contain adventitious sounds emanating from the subject and/or sound signal components which have been introduced to the respiratory system of the subject (e.g. by introduction of a sound signal into the subject's airway via the nose/mouth) and transmitted through at least part of the respiratory system to T1 and/or T2. Signals from the analogue-to-digital converter 216 (A/D) can undergo pre-processing 212 before being transmitted to transducers T1 and T2.

Parameter monitor is capable of monitoring a parameter, for example, esophageal pH using esophageal pH transducer, P. Signals from the monitor can undergo pre-processing 214 and are input via analogue-to-digital converter 216 (A/D) to processor 202 which is in communication with input device 203 and display device 204. A printer (not shown), and other electronic peripherals, may also be provided. The processor includes an identifier module 210 adapted to identify the occurrence of one or more events in the signal representing the monitored parameter. As indicated above, identification of the event(s) may be performed manually by a user using input device 203 to identify the event in the data set. This may be done by reviewing monitored parameter values or a graphical time-based representation of the data using display 204. Alternatively, identification of the event may be performed automatically by the identifier module 210, based on rules for selection programmed into the module. For example, the module may be pre-programmed to identify automatically a pH change as a reflux event. The rules may be pre-set in the system. Preferably, the rules may be added to or altered by a user via input device 203. Alternatively, a rule may be determined by a statistical evaluation of the entire monitored period. This an be done by setting a threshold value that is determined individually based on the characteristics of the entire monitored period, e.g. a heart rate threshold value may be set to occur when the heart rate of a patient at any time exceeds or falls below the 99^{th} or the 1^{st} percentile, respectively.

ARM module 220 is configured to locate one or more acoustic markers in the acoustic respiratory signal. The located marker(s) may coincide with the timing of the identified event, or may precede the event or ensue it. In some circumstances the respiratory marker will persist for a period of time including the physiological event. Thus, the marker module determines the extent of the ARMs, preferably in short intervals preceding and/or following the physiological event. Determining the extent of the ARMs may evaluate any one or combination of the amplitude, duration, frequency, number or duty cycle of the ARM. Other quantitative or semi-quantitative scores or combination of scores may be used.

Characterization module 230 characterizes the relationship between the event(s) and the one or more ARMs by determining a temporal relationship between the two. Preferably, the characterization module 230 estimates a mathematical model such as a distribution-function, representing the temporal relationship between the occurrence of the one or more events, and the ARMs identified in the acoustic signal. Parameters of the mathematical relationship can then be calculated to quantify the relationship. These parameters can be used to provide an objective assessment of the kinetics involved in the event. For example, the timing of the first moment of the distribution, or the timing of a deflection point in the distribution function.

Characterization of the relationship between one or more ARMs and a physiological event may be based on a single event occurrence. Preferably however, several events of the same type are identified and the ensemble of event data are averaged before the relationship is characterized. For example, the ARMs before, during and after each dose of medications such as Albuterol (a bronco dilator) or Lasex (a diuretic) may be monitored over a 10 day period. This may improve accuracy of the characterization. Where characterization of the relationship between the ARM and physiological event is based on graphic display of such relationships, a mathematical model or curve fitting of the ARM occurrence in each short-time interval may be determined by the characterization module. Such mathematical models may be based on e.g. an error-function or on a sigmoid function (Hill Equation) or on a frequency distribution function such as a Gaussian or gamma distribution or polynomial function or other suitable mathematical function. Such mathematical models may be averaged.

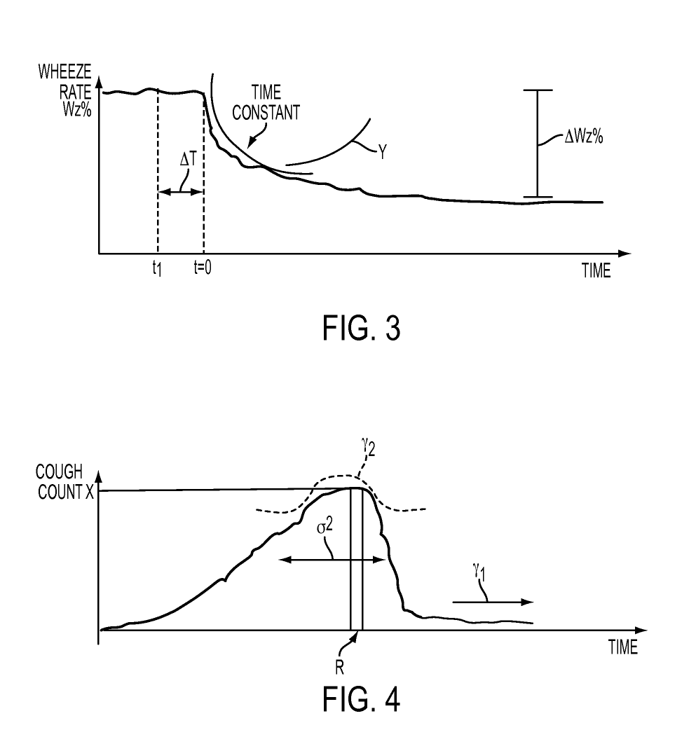

Determination of specific parameters from the graphical representations and/or the mathematical models may include, for example calculation of a step-change in absolute or relative terms (e.g. ΔWz % in FIG. 3), determination of a delay between the occurrence of a physiological event and the onset of response in the ARMs (e.g. ΔT in FIG. 3), determination of a time-constant indicative of the kinetics of the change in the ARM following the pathophysiological event (e.g. τ in FIG. 3). Alternatively/additionally, the characteristics of a distribution function representing the ARM preceding or following the events may be determined. Such characteristics may include for example the variance (e.g. σ² in FIG. 4), skewness (e.g. γ₁ in FIG. 4) or kurtosis (e.g. γ₂ in FIG. 4) and the difference between the integrated area under the curve before and after the event.

Referring now to FIG. 3 there is shown a graph representative of a subject's wheeze rate of a subject as a function of time. The wheeze rate (Wz %) is calculated as the duty cycle of wheezing time in relation to total breathing time, in a given period of monitoring (for example, one minute). An event is illustrated at t₁, involving an administration of a dose of a bronchodilator. A curve is fitted to the wheeze rate data at y and this can be represented as an exponential equation taking a form such as Equation 1.

$$y = A {e}^{- \frac{t}{τ}} \qquad Equation 1$$

Equation 1 denotes an exponential function, where t is time (t=0 being the “Response time” i.e. the time of a noticeable effect on wheeze rate) and can have only positive values. e is an exponent constant (“Euler's number”), A and τ are constants of the equation, and y is the data format/respiratory marker value, in this case the wheeze rate. Curve fitting can be done using any suitable method known in the art such as, for example Least Mean Squares method.

From this characterization, several quantitative parameters can be obtained. For example, time to effect can be determined using the time difference (ΔT) between the event E and the response time (t=0). In addition, the time constant (τ) and the difference in wheeze rate before and after the Bronchodilator dose (ΔWz %) can be used to quantify the subject's rate of response and the effectiveness of treatment respectively.

A similar example may be seen with reference to a graph (not shown) plotting data indicative of cough count as a function of time. The cough count may be calculated as the number of coughs in the monitored period (e.g. one minute). A physiological event involves administration of cough suppressor medication. The distribution function can be curve-fitted to a mathematical function such as the exponential equation depicted in Equation 1. After a time delay ΔT the cough rate decreases according to time constant τ and the difference in cough count before and after the administration of cough suppressor medication can be determined.

In another similar example, crackle count can be represented on a graph (not shown) as a function of time. The crackle count may be calculated as the number of crackles in the monitored period (e.g. one minute). An event involving the application of Positive End-Expiratory Pressure (PEEP) in a patient being mechanically ventilated is identified as a therapeutic event. Again, the distribution function can be curve-fitted to a mathematical function such as the exponential depicted in Equation 1. From this characterization, several quantitative parameters can be obtained, such as the time difference (ΔT) indicating the time delay between onset of therapy and onset of response, the time Constant τ indicating the rate of response and the difference in cough count before and after the administration of PEEP, indicating the effectiveness of the therapy.

In yet another similar example, snore rate can be presented graphically as a function of time. The snore rate can be calculated as the number of snores in a monitored period (e.g. one minute). A therapeutic event involving application of CPAP in a spontaneously breathing patient can be identified on the graph and the distribution function can be curve-fitted to a mathematical function such as the exponential depicted in Equation 1. From this characterization, several quantitative parameters can be obtained, such as the time difference (ΔT) indicating the delay between onset of therapy and the onset of a response in the patient. Time Constant τ indicates the rate of response and the difference in cough count before and after the administration of CPAP indicates the extent of improvement (reduction) in snoring. Such method has utility in determining the value of CPAP treatment in sleep apnea sufferers.

Reference is now made to FIG. 4, which illustrates a cough count graph as a function of time. The cough count is calculated as the number of coughs in a monitored period (e.g. one minute). An event R is illustrated, involving a reflux event. The distribution function can be curve-fitted to a mathematical function (shown in broken lines, no arrows), such as a Gaussian distribution. Additionally, several quantitative parameters can be obtained, such as the variance (σ²), the skewness (γ₁), and the kurtosis (γ₂).

Variance indicates is the extent of variability of the graph's value around the event R. The variance (σ²) is the mean of the squares of the distances between all the data points and the event data point. Equation 2 denotes the calculation of variance, where σ² is the variance of the graph, N is the number of data points in the graph, xᵢ is the value of data point i and x₀ is the value of the data point corresponding to the event.

$${σ}^{2} = \frac{1}{N - 1} {\sum }_{i = 1}^{N} {( {x}_{i} - {x}_{0} )}^{2} \qquad Equation 2$$

The skewness (γ₁) of the graph relates to the level of asymmetry in the graph with respect to the Event data point.

$${γ}_{1} = \frac{1}{N} {\sum }_{i = 1}^{N} {( \frac{{x}_{i} - {x}_{0}}{σ} )}^{3} \qquad Equation 3$$

Equation 3 denotes the calculation of skewness, where γ₁ is the skewness of the graph, N is the number of data points in the graph, σ is the standard deviation of the graph which is the square root of the variance of the graph (σ²) and xᵢ is the value of data point i and x₀ is the value of the data point corresponding to the event.

The kurtosis (γ₂) of the graph relate to the level of “peakedness” of the data, due to abnormal rate of occurrence of either very small or very large values in the graph.

$${γ}_{2} = \frac{1}{N} {\sum }_{i = 1}^{N} {( \frac{{x}_{i} - {x}_{0}}{σ} )}^{4} \qquad Equation 4$$

Equation 4 denotes the calculation of kurtosis, where γ₂ is the kurtosis of the graph, N is the number of data points in the graph, σ is the standard deviation of the graph which is the square root of the variance of the graph (σ²) and xᵢ is the value of data point i and x₀ is the value of the data point corresponding to the event.

In the example portrayed in FIG. 4, the graph appears to have a “negative skewness”, that is, the distribution of the coughs throughout the monitored period leans towards higher values preceding the event R. This is typical of a “Cough-induced Reflux”, where the coughs lead (and possibly cause) the reflux event.

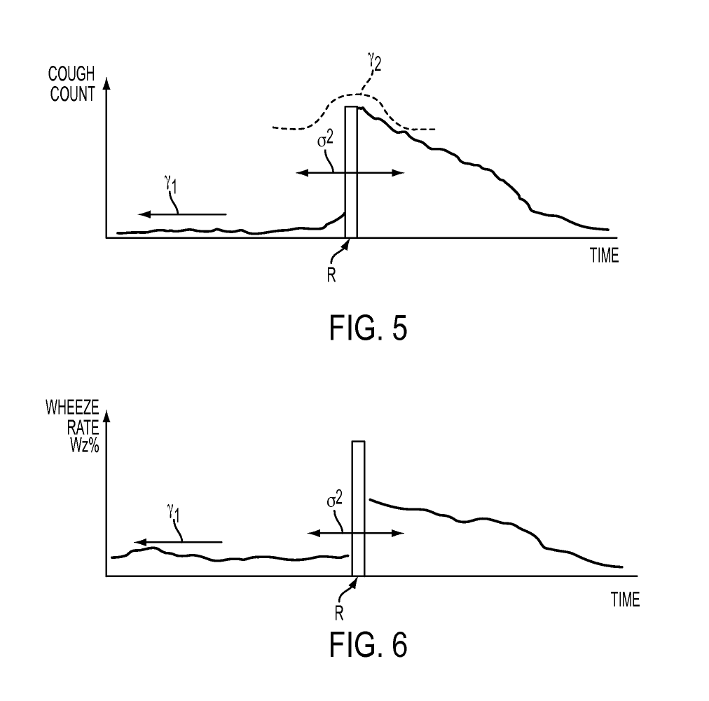

Reference is now made to FIG. 5, which illustrates a cough count graph as a function of time. The cough count is calculated as the number of coughs in a monitored period (e.g. one minute). An event R is illustrated, involving a reflux event. The distribution function can be curve-fitted to a mathematical function (shown in broken line, no arrows), such as a Gaussian distribution. Additionally, several quantitative parameters can be obtained, such as the variance (σ²), the skewness (γ₁), and the kurtosis (γ₂). The variance can be calculated as depicted in Equation 2, the skewness can be calculated as depicted in Equation 3 and the kurtosis can be calculated as depicted in Equation 4.

In the example portrayed in FIG. 5, the graph appears to have a “positive skewness”, that is, the distribution of the coughs throughout the monitored period leans towards higher values ensuing the event R. This is typical of “Reflux-induced Coughs”, where the reflux event leads (and possibly causes) the rise in coughs.

Reference is now made to FIG. 6, which illustrates a wheeze rate graph as a function of time. The wheeze rate (Wz %) is calculated as the duty cycle of wheezing time in relation to total breathing time, in the monitored period (e.g., one minute). An identified event R involves a reflux event. The distribution function can be curve-fitted to a mathematical function. Additionally, several quantitative parameters can be obtained, such as the variance (σ²) and the skewness (γ₁). The variance can be calculated as depicted in Equation 2, and the skewness can be calculated as depicted in Equation 3.

In the example portrayed in FIG. 6, the graph appears to have a “positive skewness”, that is, the distribution of wheezes (as shown by the wheeze rate) throughout the monitored period leans towards higher values ensuing the event. This is typical of “Reflux-induced Wheezes”, where the reflux event leads (and possibly causes) the rise in wheeze rate.

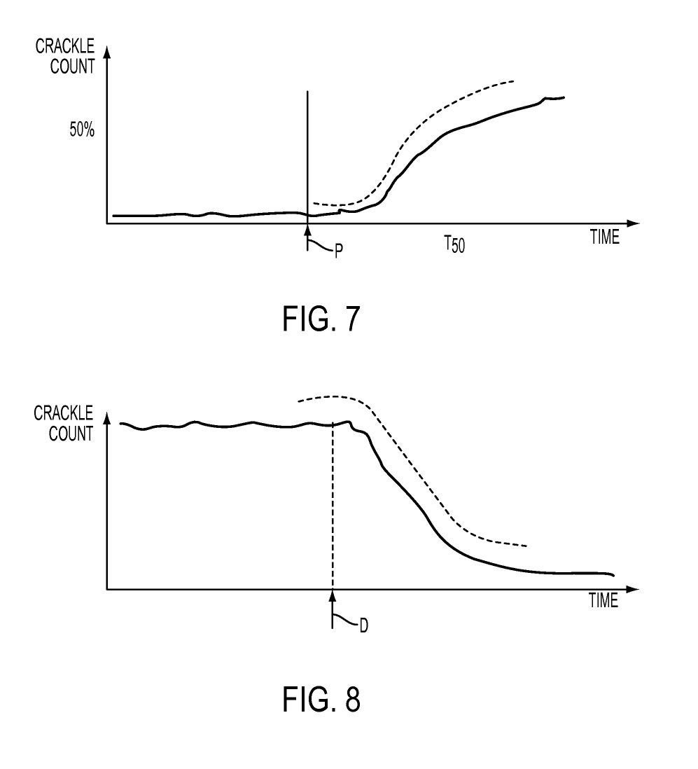

Reference is now made to FIG. 7, which illustrates a crackle count graph as a function of time. The crackle count is calculated as the number of crackles in the monitored period (e.g., one minute). An event P is illustrated, involving a change in a posture of a patient, from upright to supine position. The change in posture event may be identified using any suitable mechanism or means e.g. pressure and/or temperature sensors arranged between the subject and mattress. The distribution function can be curve fitted to a mathematical function (shown in broken lines), such as a Hill Equation, Error Function, and Polynomial Fit etc.

Equation 5 denotes a Hill Equation, where t stands for time (t=0 being the time of the event—in this case, the change in posture), and can have only positive values. θ is the fraction of the maximum data, in this case a fraction of the maximum amount of crackles appearing in the graph, T₅₀ is the time where the data is 50% of the maximum value and n is a Hill equation exponent which determines the acuteness of the change in the ARMs. m is a proportion coefficient.

$$Θ = m \frac{{( t )}^{n}}{{( t )}^{n} + {( {T}_{50} )}^{n}} \qquad Equation 5$$

Equation 6 denotes an Error Function, where t stands for time (t=0 will be determined during the curve-fitting). π is the “pi” constant, e is the exponential constant and y is the data format, in this case the crackle count, and k is the variable of integration.

$$y ( t ) = \frac{2}{π} {\int }_{0}^{t} {e}^{- {k}^{2}} d k \qquad Equation 5$$

Reference is now made to FIG. 8, which illustrates a crackle count graph as a function of time. The crackle count is calculated as the number of crackles in a monitored period (e.g. one minute). An event D is illustrated, involving an administration of diuretic medication. The distribution function can be curve-fitted to a mathematical function (shown in broken line) such as Hill Equation, Error Function, and Polynomial Fit etc. A Hill Equation is depicted in Equation 5, and an Error Function is depicted in Equation 6.

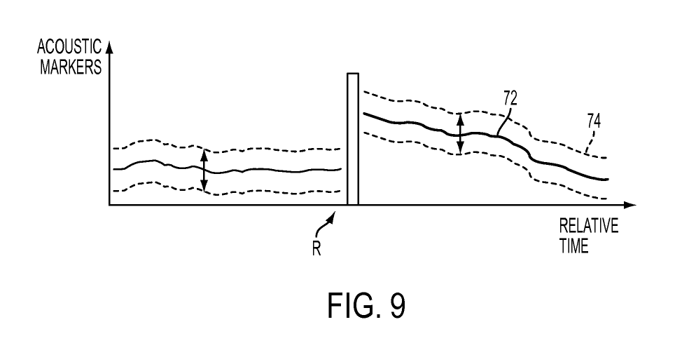

Reference is now made to FIG. 9, which illustrates how multiple events can be displayed on a single plot. The event R, in this case a Reflux Event, is shown at the middle of the graph, while the acoustic markers are mapped around the event on a “relative time” axis, which can be linear or logarithmic. Examples of acoustic markers may include Wheeze Rate, Cough Count, Crackle Count and Snore Rate to name a few. The value of the acoustic markers is arranged and displayed as a solid line at 72 with error intervals shown at 74. These intervals may represent the Standard Deviation of the acoustic markers.

The present disclosure provides a method for recognizing in a sequence of events the correlation between acoustic markers and e.g. pathophysiological events which may provide diagnostic information on a subject's condition. Various embodiments facilitate quantitative analysis.

For example, an asthma patient may show a positive response to a bronchodilator such as ventoline, determined by the diminution in wheezing as detected by auscultation. The diminution in wheezing indicates reversibility of airway obstruction. However, prior to the present invention it has not been feasible to ascertain the quantitative kinetics of this response. Likewise, the prior art has failed to provide a method, apparatus or system for ascertaining a causal or at least a temporal link between e.g. a reflux event and an ARM. Similarly, other temporal correlations between e.g. physiological, pathophysiological and therapeutic events and lung sounds have not been characterized by quantitative, objective methods.

The present disclosure provides a novel approach to identifying and optionally quantifying temporal correlations between ARMs and other events that are either naturally occurring or purposefully induced in the subject. This approach has advantages in medicine where it is necessary to identify and preferably quantify cause-and-effect relationships between physiological events in order to positively diagnose a condition of a patient or subject. Alternatively or additionally, this approach may provide utility in evaluating the effectiveness of a medical intervention in a quantifiable and repeatable manner.

For example, knowing if a reflux event precedes, on average, the emergence of wheeze or cough, can lead to a diagnosis of reflux-induced asthma. Meanwhile, if a wheeze or cough predominantly precedes a reflux event, a diagnosis of asthma-induced reflux is likely. These conditions require completely different treatment. Thus, utilizing the present disclosure to characterize the nature of the relationship between the reflux event and the respiratory marker and even more desirably, quantifying that relationship is of significance and importance.

In another example, the ability to determine if shifting a position of the patient from supine to upright poses a gradual decline in the crackle count of the patient at the bases of the lung can be utilized to evaluate if the patient is suffering from congestive heart failure (positive gravitational effects) or pneumonia/lung fibrosis (negative gravitational effects). Each of these conditions requires completely different treatment.

In another example, the ability to determine if administration of a drug such as albuterol or atrovent (atropine) affects the temporal distribution of wheezes and cough can be utilized to verify that the airway narrowing manifested by wheezes is reversible (positive effect) which is, by definition, asthma. Alternatively, if there is no effect (negative response) a non-asthma obstructive airway disease may be diagnosed (e.g. COPD, bronchiolitis etc.).

In another example, the present disclosure may be used to determine if inhalation of small doses of airway irritants such as hypertonic saline or capsaicin in induces single or multiple bouts of cough. This in turn may be used to determine if a patient has a tendency for chronic cough which requires specific treatment.

It is to be understood that various modifications, additions and/or alterations may be made to the parts previously described without departing from the ambit of the present invention as defined in the claims appended hereto. Future patent applications may be filed in Australia or overseas on the basis of or claiming priority from the present application. It is to be understood that the following provisional claims are provided by way of example only, and are not intended to limit the scope of what may be claimed in any such future application. Features may be added to or omitted from the provisional claims at a later date so as to further define or re-define the invention or inventions.

1. A method for characterizing a temporal relationship between an acoustic respiratory marker and an event in a subject, including the steps of:
(a) simultaneously monitoring an acoustic signal from the respiratory system of the subject and at least one parameter selected from a group including a physiological parameter, a pathophysiological parameter, a patient-reported symptom and a therapeutic parameter associated with the subject;
(c) identifying the event in the monitored parameter(s); and
(d) identifying, in the monitored acoustic signal, the presence of one or more respiratory markers coinciding with and/or preceding and/or ensuing the event;
wherein the relationship is characterized by determining a temporal correlation between the event and the one or more acoustic respiratory markers.

2. A method according to claim 1 wherein the acoustic respiratory marker is selected from the group including but not limited to a signal indicative of a wheeze, cough, snore, crackle and a breath sound amplitude.

3. A method according to claim 1 further including the step of introducing a sound signal having known acoustic characteristics into the respiratory system of the subject and wherein the monitored acoustic signal includes the introduced sound signal after it has travelled through at least part of the respiratory system of the subject.

4. A method according to claim 1 wherein the event is selected from a group including: a reflux event, the onset or end of apnea, change of settings on a ventilator, a postural change, an indication of a symptom of a patient and administration of therapeutic agent or treatment.

5. A method according to claim 1 including the step of representing the monitored acoustic signal for a time period using a mathematical model and evaluating one or more parameters of the model, said one or more evaluated parameters being quantitative indicators of the relationship between the event and the one or more acoustic markers.

6. A method according to claim 5 wherein the time period commences during or after the event.

7. A method whereby one or more of the following are used to characterize the relationship or the coefficients of a polynomial equation:
(a) time constant, τ determined using the relationship
$$y = A {e}^{- \frac{t}{τ}} ;$$
(b) variance, σ² as determined using the relationship
$${σ}^{2} = \frac{1}{N - 1} {\sum }_{i = 1}^{N} {( {x}_{i} - {x}_{0} )}^{2} ;$$
(c) skewness, γ₁ as determined using the relationship
$${γ}_{1} = \frac{1}{N} {\sum }_{i = 1}^{N} {( \frac{{x}_{i} - {x}_{0}}{σ} )}^{3} ;$$
(d) kurtosis, γ₂ as determined using the relationship
$${γ}_{2} = \frac{1}{N} {\sum }_{i = 1}^{N} {( \frac{{x}_{i} - {x}_{0}}{σ} )}^{4} ;$$
and
(e) Hill equation exponent, n as determined using the relationship
$$Θ = m \frac{{( t )}^{n}}{{( t )}^{n} + {( {T}_{50} )}^{n}} ;$$
where:
y is the respiratory marker value;
A and τ are constants;
N is the number of acoustic signal data points;
σ is the standard deviation;
xᵢ is the value of data point i
x₀ is the value of the data point corresponding to the event;
θ is the fraction of the maximum data;
T₅₀ is the time where the data is 50% of the maximum value;
t is the time;
n is an exponent representing acuteness of the change in respiratory marker; and
m is a proportion coefficient.

8. A method according to claim 5 wherein the time period commences before the event.

9. A method according to claim 5 including the step of averaging data representing the acoustic signal for a plurality of events identified in the monitored parameter to characterize the relationship.

10. A method according to claim 5 including the step of determining the extent of the respiratory marker.

11. A method for diagnosing a condition in the subject including the characterizing method according to claim 5.

12. A system for characterizing a relationship between an acoustic respiratory marker from a subject and an event in a subject, the system including:
(a) acoustic monitoring means for monitoring an acoustic signal from the respiratory system of the subject;
(b) parameter monitoring means for monitoring a parameter selected from the group including a physiological parameter, a pathophysiological parameter, a therapeutic parameter associated with the subject and a patient-reported symptom; and
(c) processing means incorporating:
(i) an identifier module identifying the occurrence of the event in the monitored parameter(s);
(ii) a marker module locating one or more acoustic markers in the acoustic respiratory signal coinciding with and/or preceding and/or ensuing the event; and
(iii) a characterization module configured to characterize the relationship by determining a temporal correlation between the event and the one or more acoustic respiratory markers.

13. A system according to claim 12 further including a sound source generating a sound signal having known characteristics and means for introducing the sound signal to the respiratory system of the subject, wherein the acoustic monitoring means monitors the introduced sound signal after it has travelled though at least part of the respiratory system of the subject.

14. A system according to claim 12 further including a user interface presenting a graphical display of the monitored signals and receiving a user selection of a data window for further characterization.

15. A system according to claim 12 wherein the characterizing module further characterizes the relationship by calculating a mathematical model approximating at least a portion of the monitored acoustic signal.

16. A system according to claim 15 wherein the characterizing module further evaluates parameters of the mathematical model to quantify the characterization.

17. A system according to claim 12 wherein the acoustic respiratory marker is selected from the group including but not limited to a signal indicative of a wheeze, cough, snore, crackle and a breath sound amplitude.

18. A system according to claim 12 wherein the event is selected from the group including but not limited to: a reflux event, the onset or end of apnea, change of settings on a ventilator, a postural change and administration of a therapeutic agent or treatment.

19. (canceled)

20. (canceled)
